(19) United States
(12) Patent Application Publication
(10) Pub. No.: US 2023/0329570 A1
(43) Pub. Date: Oct. 19, 2023

(54) ELECTRONIC DEVICE, STORAGE MEDIUM FOR ELECTRONIC DEVICE, AND CONTROL METHOD FOR ELECTRONIC DEVICE

(71) Applicant: CASIO COMPUTER CO., LTD., Shibuya-ku, Tokyo (JP)

(72) Inventors: Takahiro TOMIDA, Tokyo (JP); Toshihiko OTSUKA, Tokyo (JP)

(73) Assignee: CASIO COMPUTER CO., LTD., Shibuya-ku, Tokyo (JP)

(21) Appl. No.: 18/026,564

(22) PCT Filed: Jul. 19, 2021
(86) PCT No.: PCT/JP2021/027013

§ 371 Date: Mar. 15, 2023

(30) Foreign Application Priority Data
Sep. 23, 2020 (JP) 2020-158423

(51) Int. Cl. A61B 5/026; A61B 5/00

(52) U.S. Cl. CPC A61B 5/026; A61B 5/0077; A61B 5/7264

(57) ABSTRACT

An electronic device includes at least one processor to execute processing including: acquiring first pulse wave information indicating a pulse wave from first video obtained by imaging at least a part of a body and acquiring second pulse wave information indicating a pulse wave from second video obtained by imaging a part of the body or a part corresponding to the part of body; acquiring, from the first and second pulse wave information, a baseline as an average value of the pulse wave and a pulse wave amplitude as an average amplitude of the pulse wave in a predetermined period of time, and deriving a baseline change rate indicating a change and a pulse wave amplitude change rate indicating a change in pulse wave amplitude; and determining a blood circulation state based on a relation of the baseline change rate and the pulse wave amplitude change rate.

### TECHNICAL FIELD

The present invention relates to an electronic device, a control program for an electronic device, and a control method for an electronic device.

### BACKGROUND ART

As conventional techniques for measuring blood flow of skin surface, for example, a laser Doppler method and a laser speckle method are known.

In the laser Doppler method, the blood flow is measured by utilizing a frequency shift that occurs when laser light irradiated onto the skin surface is reflected by red blood cells moving inside capillaries. A blood flow rate can be calculated because the percentage of light shifted is proportional to the number of red blood cells, and the magnitude of the shift is proportional to the blood flow velocity. In the laser speckle method, the blood flow is measured by utilizing a granular pattern referred to as a speckle pattern, which is observed as a result of overlapping of scattered light returned when in-phase light such as laser light is irradiated onto a group of scattering particles such as biological tissue. The speckle pattern dynamically changes in pattern as red blood cells move inside capillaries. A blood flow rate can therefore be calculated from this change. Examples of measurement technologies involving the use of the laser speckle method include a blood flow imager disclosed in Non-Patent Document 1.

A device for extracting a pulse wave through video analysis is also known. This type of technology is disclosed, for example, in Patent Document 1. Patent Document 1 discloses a pulse wave velocity measurement method including: an imaging step of simultaneously imaging two mutually different parts from among plural parts of a human body in a non-contact state by a single visible light camera and generating continuous time series image data; a pulse wave detection step of detecting each pulse wave in the two different parts of the human body from the image data based on a temporal change in pixel value of the two different parts of the human body; and a pulse wave velocity calculation step of calculating a pulse wave velocity of the human body based on a time difference between pulse waves in the two different parts of the human body.
Non-Patent Document 1: OMEGAWAVE, INC., [online], [retrieved on Sep. 16, 2020], Internet <http://www.omegawave.co.jp/products/oz/principle.shtml>
Patent Document 1: Japanese Patent No. 6072893

### DISCLOSURE OF THE INVENTION

### Problems to be Solved by the Invention

However, with the methods in which laser reflection is utilized, it is possible to calculate a blood flow rate per unit time in a unit weight of tissue, but it is difficult to distinguish between different hemodynamic states such as congestive and hyperemic states. Likewise, with the conventional technology for extracting a pulse wave through video analysis, it is difficult to distinguish between hemodynamic states such as congestive and hyperemic states as well as measure blood flow.

An object of the present invention is to provide an electronic device, a control program for an electronic device, and a control method for an electronic device with which it is possible to measure blood flow based on video captured using a general camera and to distinguish between different hemodynamic states such as congestive and hyperemic states.

### Means for Solving the Problems

In order to achieve the above-described object, an electronic device according to an aspect of the present invention includes: a video processing unit configured to acquire first pulse wave information indicating a pulse wave from first video obtained through imaging of a specific part of a subject's body during a first period, and acquire second pulse wave information indicating a pulse wave from second video obtained through imaging of the specific part of the subject's body during a second period later than the first period; a data processing unit configured to acquire, from the first pulse wave information and from the second pulse wave information, a baseline of the pulse wave and a pulse wave amplitude, and derive a baseline change index and a pulse wave amplitude change index, the baseline change index indicating a change in the baseline between the first pulse wave information and the second pulse wave information, the pulse wave amplitude change index indicating a change in the pulse wave amplitude between the first pulse wave information and the second pulse wave information; and an identification processing unit configured to identify a hemodynamic state based on a relationship between the baseline change index and the pulse wave amplitude change index.

### Effects of the Invention

An electronic device, a control program for an electronic device, and a control method for an electronic device according to the present invention make it possible to distinguish between different hemodynamic states such as congestive and hyperemic states.

### BRIEF DESCRIPTION OF THE DRAWINGS

FIG. 1 is a configuration diagram illustrating a configuration of a measurement system according to one embodiment of the present invention;

FIG. 2 is a configuration diagram illustrating an external configuration of an electronic device and an imaging unit according to the one embodiment of the present invention;

FIG. 3 is a block diagram illustrating a hardware configuration of the electronic device according to the one embodiment of the present invention;

FIG. 4 is a configuration diagram illustrating an external configuration of the front of an electronic device according to another embodiment different from that illustrated in FIG. 2;

FIG. 5 is a configuration diagram illustrating an external configuration of the back of the electronic device according to the embodiment different from that illustrated in FIG. 2;

FIG. 6 is a functional block diagram illustrating elements of a functional configuration of the electronic device according to the one embodiment of the present invention that perform measurement processing;

FIG. 7 is a graph showing temporal change in converted luminance before cold water loading as measured using the electronic device according to the one embodiment of the present invention;

FIG. 8 is a graph showing temporal change in the converted luminance after the cold water loading as measured using the electronic device according to the one embodiment of the present invention;

FIG. 9 is a graph schematically showing pulse wave amplitude as measured using the electronic device according to the one embodiment of the present invention;

FIG. 10 is a graph showing, in an enlarged manner, the waveform of the temporal change in the converted luminance before the cold water loading as measured using the electronic device according to the one embodiment of the present invention;

FIG. 11 is a graph showing, in an enlarged manner, the waveform of the temporal change in the converted luminance after the cold water loading as measured using the electronic device according to the one embodiment of the present invention;

FIG. 12 is a graph showing temporal change in the converted luminance of a target site on a left arm as measured using the electronic device according to the one embodiment of the present invention;

FIG. 13 is a graph showing temporal change in the converted luminance of a target site on a right arm given a vaccination, as measured using the electronic device according to the one embodiment of the present invention;

FIG. 14 is a graph showing, in an enlarged manner, the waveform of temporal change in the converted luminance of the target site on the left arm as measured using the electronic device according to the one embodiment of the present invention;

FIG. 15 is a graph showing, in an enlarged manner, the waveform of temporal change in the converted luminance of the target site on the right arm given the vaccination as measured using the electronic device according to the one embodiment of the present invention;

FIG. 16 is a table showing results of comparisons by the electronic device according to the one embodiment of the present invention between before and after the cold water loading, and between the target site with the vaccination and the target site without the vaccination;

FIG. 17 is a graph based on measurement results from the electronic device according to the one embodiment of the present invention, in which the horizontal axis represents baseline change rate and the vertical axis represents pulse wave amplitude change rate;

FIG. 18 is a graph showing criteria for identifying a hemodynamic state using the electronic device according to the one embodiment of the present invention;

FIG. 19 is a diagram showing an example of a measurement result displayed on a display unit of the electronic device according to the one embodiment of the present invention;

FIG. 20 is a flowchart for describing a flow of the first half of the measurement processing that is performed by the electronic device according to the one embodiment of the present invention;

FIG. 21 is a flowchart for describing a flow of the latter half of the measurement processing that is performed by the electronic device according to the one embodiment of the present invention;

FIG. 22 is a schematic diagram showing an experiment conducted using the electronic device according to the one embodiment of the present invention;

FIG. 23 is an image captured when a hand was kept at a lower position and measured using a two-dimensional laser blood flowmeter according to a comparative example;

FIG. 24 is an image captured when the hand was kept at a higher position and measured using the two-dimensional laser blood flowmeter according to the comparative example;

FIG. 25 is a graph showing temporal change in the converted luminance corresponding to a case where the hand was kept at the lower position and measured using the electronic device according to the one embodiment of the present invention;

FIG. 26 is a graph showing temporal change in the converted luminance corresponding to a case where the hand was kept at the higher position and measured using the electronic device according to the one embodiment of the present invention;

FIG. 27 is a graph showing, in an enlarged manner, the waveform of the temporal change in the converted luminance corresponding to the case where the hand was kept at the lower position and measured using the electronic device according to the one embodiment of the present invention; and

FIG. 28 is a graph showing, in an enlarged manner, the waveform of the temporal change in the converted luminance corresponding to the case where the hand was kept at the higher position and measured using the electronic device according to the one embodiment of the present invention.

### PREFERRED MODE FOR CARRYING OUT THE INVENTION

The following describes an embodiment of the present invention with reference to the drawings.

### [Overview of Embodiment]

An electronic device 1 according to an embodiment of the present invention is a measurement device that measures a hemodynamic state based on video obtained through imaging of a measurement site of a user.

### [System Configuration]

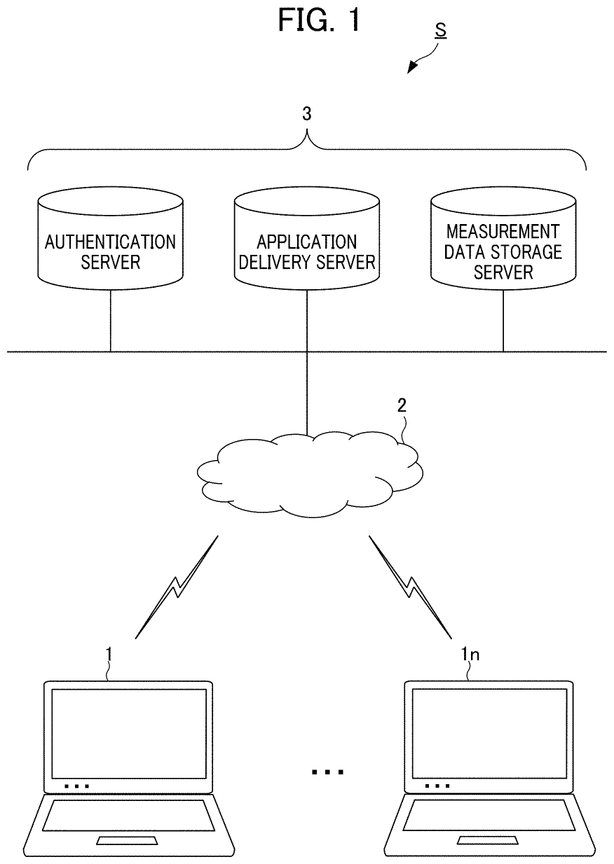

FIG. 1 is a block diagram illustrating an overall configuration of a measurement system S including the electronic device 1 according to the present embodiment. As illustrated in FIG. 1, the measurement system S includes a plurality of electronic devices 1, a network 2, and a server group 3. No particular limitations are placed on the number of electronic devices 1, and the measurement system S may include n (n is a natural number) electronic devices 1. In the following description, each electronic device 1 is simply referred to as “the electronic device 1” by omitting a letter at the end of a reference numeral thereof, where there is no particular need to distinguish between the n electronic devices 1 for the description.

The electronic device 1 is a computer that measures a hemodynamic state of a user based on video. The electronic device 1 is communicatively connected to servers included in the server group 3 via the network 2.

The network 2 is implemented by, for example, the Internet, a local area network (LAN), a cellular phone network, or a combination of any of these networks.

The server group 3 includes various servers that operate in conjunction with the electronic device 1. For example, the server group 3 includes an authentication server for authentication of the user of the electronic device 1. For another example, the server group 3 includes an application delivery server that delivers application software for implementing functions of the electronic device 1. For another example, the server group 3 includes a measurement data storage server that stores user profile information, which is information including setting information related to the user, a history of usage of the electronic device 1 by the user, and the like.

It should be noted that the measurement system S shown in FIG. 1 is merely an example, and the server group 3 may include a server having another function. The plurality of servers included in the server group 3 may be implemented by separate server devices or by a single server device.

### [Electronic Device]

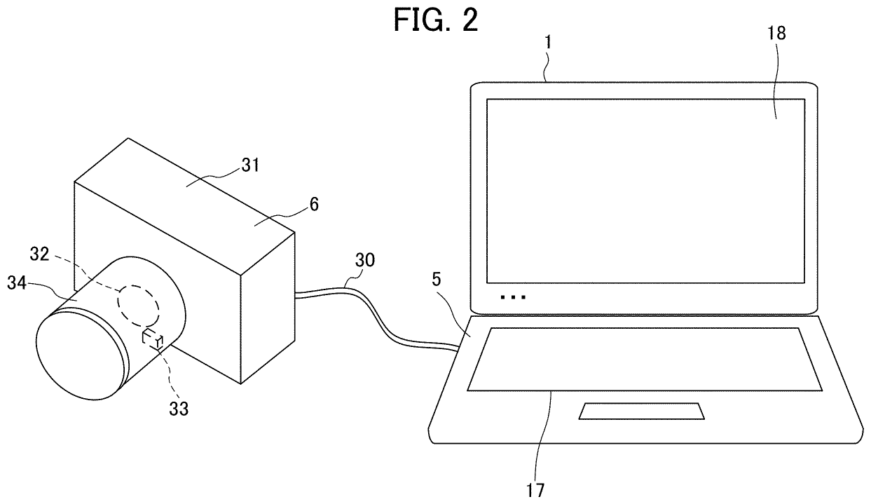

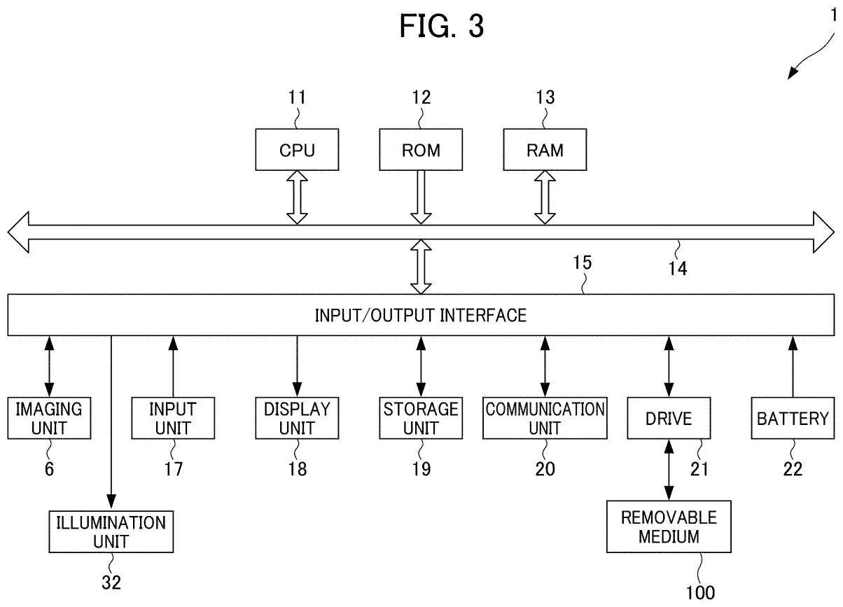

Referring to FIGS. 2 and 3, the following describes examples of the electronic device 1 and an imaging unit 6. FIG. 2 is a configuration diagram illustrating an external configuration of the electronic device 1 and the imaging unit 6 according to one embodiment of the present invention. FIG. 3 is a block diagram illustrating a hardware configuration of the electronic device 1 according to the one embodiment of the present invention.

As illustrated in FIGS. 2 and 3, the electronic device 1 includes a housing 5, a central processing unit (CPU) 11, read only memory (ROM) 12, random access memory (RAM) 13, a bus 14, an input/output interface 15, the imaging unit 6, an input unit 17, an output unit 18, a storage unit 19, a communication unit 20, a drive 21, and a battery 22.

The housing 5 shown in FIG. 2 contains various electronic components. As shown in FIG. 2, the electronic device 1 according to the present embodiment is a notebook computer, and the housing 5 is foldable.

The CPU 11 shown in FIG. 3 is a processor that executes various processes in accordance with programs recorded in the ROM 12 or programs loaded into the RAM 13 from the storage unit 19.

The RAM 13 also stores, as appropriate, other data such as data necessary for the CPU 11 to perform various processes.

The CPU 11, the ROM 12, and the RAM 13 are connected to each other via the bus 14. The input/output interface 15 is also connected to the bus 14. The input unit 17, the output unit 18, the storage unit 19, the communication unit 20, the drive 21, and the battery 22 are connected to the input/output interface 15.

The input unit 17 receives operations inputted by the user. The input unit 17 is, for example, implemented by a plurality of buttons or a keyboard.

The output unit 18 displays various information thereon to present the various information to the user. The output unit 18 includes, for example, a liquid crystal display and displays an image based on image data outputted by the CPU 11.

The storage unit 19 includes semiconductor memory such as dynamic random access memory (DRAM) and stores therein various data.

The communication unit 20 performs communication control for the CPU 11 to communicate with other devices (e.g., the servers included in the server group 3) via the network 2.

The drive 21 includes an interface that can receive placement of a removable medium 100. The removable medium 100, which is a magnetic disk, an optical disk, a magneto-optical disk, semiconductor memory, or the like, is placed into the drive 21 as appropriate. The removable medium 100 stores therein programs for performing composite display processing described below and various data such as image data. The programs and various data such as image data read from the removable medium 100 by the drive 21 are installed in the storage unit 19 as needed.

The battery 22 is configured to supply electric power to other components and to be rechargeable by being connected to an external power source. When the electronic device 1 is not connected to an external power source, the electronic device 1 operates on the electric power from the battery 22.

### [Imaging Unit]

The imaging unit 6 is used to capture video of a subject and is electrically connected to the electronic device 1. FIG. 2 shows an example in which the imaging unit is connected to the input/output interface 15 of the electronic device 1 via a connector 30 such as a USB connector. However, the imaging unit is not limited to being connected in a wired manner and may be connected wirelessly.

The imaging unit 6 has a body unit 31, a cover 34 disposed on the front side of the body unit 31, an optical lens unit 32 disposed inside the cover 34, and an illumination unit 33 disposed inside the cover 34.

The body unit 31 contains, for example, an image sensor. The image sensor contained in the body unit 31 includes a photoelectric conversion element, an analog front end (AFE), and other components. The photoelectric conversion element includes, for example, a complementary metal oxide semiconductor (CMOS) photoelectric conversion element. A subject image enters the photoelectric conversion element from the optical lens unit. The photoelectric conversion element then photoelectrically converts the subject image (imaging), accumulates image signals for a certain period of time, and supplies the accumulated image signals to the AFE as analog signals in sequence. The AFE performs various types of signal processing, such as analog/digital (A/D) conversion processing, on the analog image signals. As a result of the various types of signal processing, digital signals are generated and outputted as output signals of the imaging unit 6. Such output signals of the imaging unit 6 are supplied to, for example, the CPU 11 as appropriate. The body unit 31 also contains peripheral circuitry for adjusting setting parameters such as focus, exposure, and white balance as needed.

The optical lens unit 32 includes a condenser lens, such as a focus lens or a zoom lens, for imaging of a subject. The illumination unit 33 includes an LED.

The cover 34 is disposed on the front side of the body unit 31 and is cylindrical in shape. The optical lens unit 32 and the illumination unit 33 according to the present embodiment are disposed inside the cover 34.

A distal end of the cover 34 of the imaging unit 6 is pressed against the subject's skin, so that the effects of external light can be reduced. This manner makes it possible to avoid a situation where the distance from the optical lens unit 32 to the measurement site is not constant due to, for example, the subject's body movements and a situation where illumination conditions are not constant due to, for example, changes in brightness. This manner helps effectively reduce the effects of changes in ambient brightness and easily maintain a constant positional relationship between the optical lens unit 32 and the subject, significantly improving measurement accuracy and stability.

The configuration of the electronic device 1 and the imaging unit 6 has been described above. However, the above-described configuration is merely an example. For example, the imaging unit 6 may include a general-purpose camera or a web camera, and the electronic device 1 may include a tablet or the like other than a notebook computer.

### [Second Electronic Device]

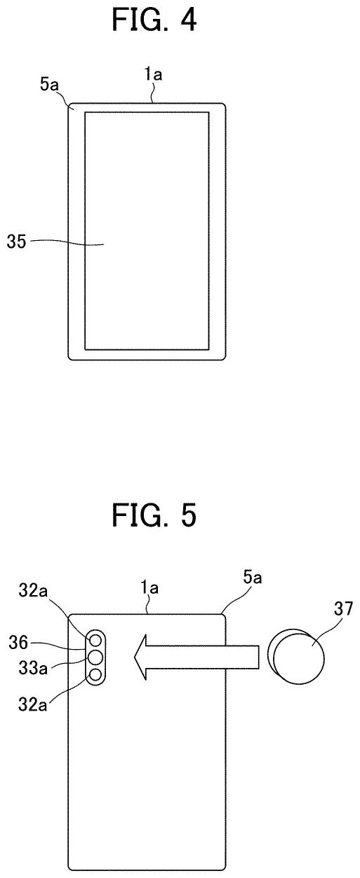

Referring to FIGS. 4 and 5, the following describes an electronic device 1a having a different configuration from the electronic device 1 described above. FIG. 4 is a configuration diagram illustrating an external configuration of the front of the electronic device 1a according to another embodiment different from that illustrated in FIG. 2. FIG. 5 is a configuration diagram illustrating an external configuration of the back of the electronic device 1a.

The electronic device 1a shown in FIGS. 4 and 5 is a smartphone. The electronic device 1a has substantially the same hardware configuration as the electronic device 1, which is a notebook computer. That is, a housing 5a of the electronic device 1a contains substantially the same electronic components as those described with reference to FIG. 3. For example, as shown in FIG. 4, a touch panel display 35 is disposed on the front side of the housing 5a of the electronic device 1a, and the touch panel display 35 functions as the input unit 17 and the output unit 18 in FIG. 3. As shown in FIG. 5, a built-in camera 36 is held integrally on the back side of the housing 5a. A macro lens 37 is attached to the built-in camera 36, so that the built-in camera 36 functions as the imaging unit 6 in FIG. 3. It should be noted that the built-in camera 36 has a plurality of lenses 32a, which correspond to the optical lens unit 32, and a light unit 33a, which corresponds to the illumination unit 33.

### [Functional Configuration]

The following describes a functional configuration of the electronic device 1 or the electronic device 1a described above. FIG. 5 is a functional block diagram illustrating elements of the functional configuration of the electronic device 1 that perform measurement processing. The measurement processing refers to a series of processes to be performed by the electronic device 1 to display measurement results based on changes in biological information values acquired from the user.

As shown in FIG. 5, a video processing unit 111, a display processing unit 112, an input processing unit 113, a data processing unit 114, an identification processing unit 115, and a communication processing unit 116 function in the CPU 11 serving as a control unit. The following describes each of the elements of the functional configuration.

The video processing unit 111 is responsible for a video processing function of processing video captured by the imaging unit 6 and extracting pulse wave information indicating a pulse wave from the video. In order to measure a change in hemodynamic state, the video processing unit 111 acquires pulse wave information from first video obtained through imaging of the user in one state and acquires pulse wave information from second video obtained through imaging of the user in another state. For example, the one state is a pre-event state and the other is a post-event state.

Examples of events include: various beauty-related treatments such as massage to stimulate blood flow and application of skin cream that acts to promote blood circulation; various activities that are expected to change blood flow such as sports, relaxation, and other physical activities; and medical procedures such as vaccination.

The display processing unit 112 is responsible for a display processing function of performing, for example, processing for generating information to be displayed on the output unit 18. The display processing unit 112 outputs, to the output unit 18, the results of comparisons between pre-event pulse wave information and post-event pulse wave information as measurement results. The measurement results to be outputted to the output unit 18 may include information indicating a hemodynamic state and a moving hue image in which blood flow variations are dynamically visualized. In the moving hue image, for example, the measurement site is divided into square subregions, and blood flow variations in each subregion are represented by hue changes.

The input processing unit 113 processes operations inputted by the user. The data processing unit 114 is responsible for an input processing function of performing image processing and other processing on various data necessary for video analysis and the like. The communication processing unit 116 then performs processing for communicating with a device such as the server group 3 on the cloud.

The data processing unit 114 is responsible for a data processing function of acquiring pulse wave information of the user in the video acquired by the video processing unit 111. According to the present embodiment, the data processing unit 114 derives a relative change (difference) from the pre-event pulse wave information and the post-event pulse wave information of the same site of the same user. The data processing unit 114 also derives a relative change (difference) from pulse wave information of a certain site (e.g., right arm) of the user and pulse wave information of a corresponding site (e.g., left arm) of the same user.

The identification processing unit 115 is responsible for an identification processing function of performing processing for identifying a hemodynamic state based on the relative change derived by the data processing unit 114. The processing for identifying a hemodynamic state by the data processing unit 114 and the identification processing unit 115 is described below.

The communication processing unit 116 communicates, for example, with the authentication server included in the server group 3. Through this communication, authentication of the user attempting display processing is performed. The communication processing unit 116 also communicates, for example, with the measurement data storage server included in the server group 3, and thus updates profile information of the user in the display processing.

### [Video Analysis]

The following describes video analysis. The video processing unit 111 acquires information on blood flow such as a pulse rate and a pulse wave using high green light absorbing properties of hemoglobin in blood. The wavelength of green signals is generally said to be 495 nm to 570 nm, and hemoglobin has higher absorption coefficients at wavelengths of around 550 nm to 660 nm. When the blood flow increases, the blood volume of the skin surface increases and the amount of hemoglobin per unit time increases. As a result, a greater amount of green signal is absorbed by hemoglobin than before the blood flow increases. This means that the luminance of a green signal to be detected decreases as the blood flow increases.

The video processing unit 111 acquires the luminance of the green signal every unit time to acquire temporal change in the luminance of the green signal. The unit time is, for example, the frame rate of a moving image, so that the luminance of the green signal can be acquired for each of the temporally successive images that form video. Preferably, an RGB filter is placed in front of an imaging element of the imaging unit 6, and the luminance value of each of RGB pixels is derived. In this case, light that has passed through the green filter is detected for a luminance value. Even if the sensitivity of the imaging element is flat with respect to the wavelength, the filter helps narrow the band of wavelengths to some extent, and thus the green signal (green light) can be detected with high accuracy.

According to the present embodiment, in order to make it easier to intuitively recognize an increase in the blood flow, a conversion process is performed so that the luminance value increases with an increase in the blood flow. More specifically, in a case where the luminance of the green signal is detected using an image sensor with an 8-bit output for each of RGB colors, a luminance is calculated by subtracting the luminance value of the detected green signal from a maximum luminance value of 255. The thus calculated luminance is used as a converted luminance.

The converted luminance can be derived by various methods such as mode, median, and average methods in order to reflect the luminance of the green signal at a plurality of locations in the measurement site. For example, an average of green signal values of all pixels in the range of the measurement site is acquired as a converted luminance every unit time, and time series information of the thus extracted converted luminance is used as pulse wave information.

The data processing unit 114 acquires, from the pulse wave information (converted luminance), an average of converted luminance values acquired during a specific period of time (predetermined period of time). In the following description, the average of the converted luminance values acquired during the specific period of time is referred to as a baseline. The data processing unit 114 also acquires an amplitude of the converted luminance from the pulse wave information (converted luminance). In the following description, the amplitude of the converted luminance acquired during the specific period of time is referred to as a pulse wave amplitude.

The data processing unit 114 according to the present embodiment identifies a hemodynamic state based on a change in the baseline and a change in the pulse wave amplitude between before and after an event. The following describes specific examples of the change in the baseline and the change in the pulse wave amplitude.

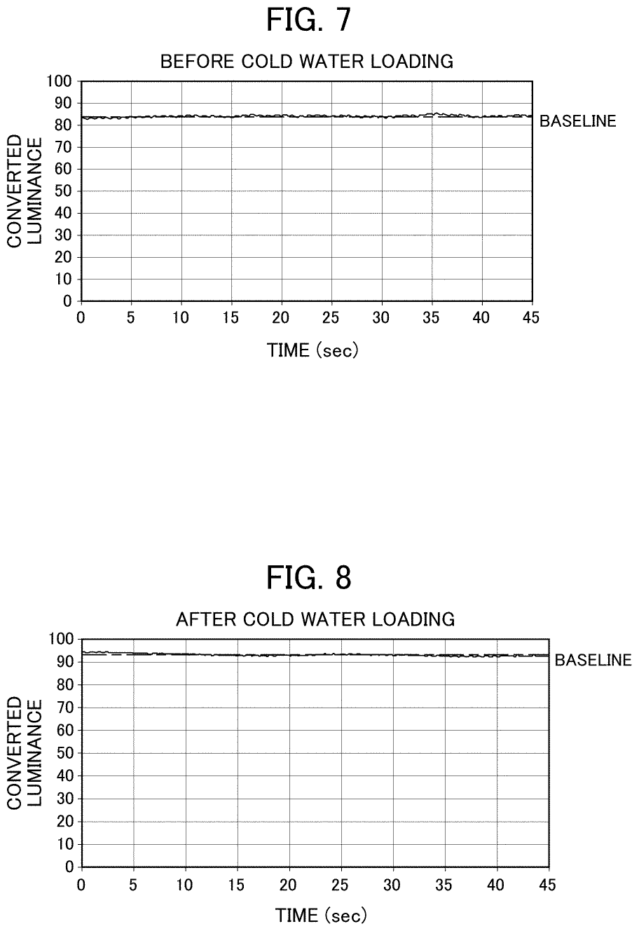

First, an exemplary change in the baseline will be described with reference to FIGS. 7 and 8. FIG. 7 is a graph showing temporal change in the converted luminance before cold water loading as measured using the electronic device 1 according to the one embodiment of the present invention. FIG. 8 is a graph showing temporal change in the converted luminance after the cold water loading as measured using the electronic device 1 according to the one embodiment of the present invention.

In the case of FIGS. 7 and 8, the measurement site is a palm, and the event is cold water loading on this palm. The cold water loading herein means submergence of the hand to the wrist in cold water at 15° C. for 1 minute. The vertical axes of the graphs in FIGS. 7 and 8 represent converted luminance, and the horizontal axes represent time (seconds). A comparison between FIGS. 7 and 8 indicates that the baseline, which is shown by a dashed and dotted line, in pulse wave information increased after the cold water loading.

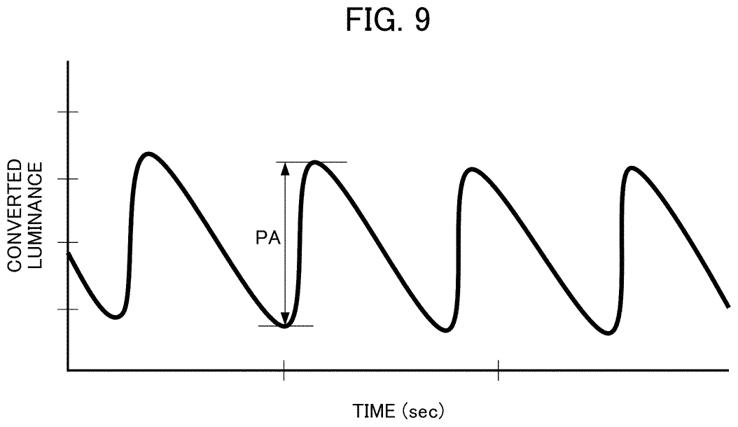

The following describes the pulse wave amplitude. FIG. 9 is a graph schematically showing the pulse wave amplitude (Pulse Amplitude; PA) as measured using the electronic device 1 according to the one embodiment of the present invention. As shown in FIG. 9, the pulse wave information resulting from the video analysis shows a periodic waveform, and the waveform is within a certain pulse wave amplitude range. This pulse wave amplitude means the difference between adjacent maximum and minimum values of a pulse wave signal.

The range for acquiring the pulse wave amplitude is preferably a region where there is no abnormal value and the amplitude is stable. For example, in a case where an abnormal value exceeding a preset threshold value is detected, the pulse wave information is acquired by avoiding inclusion of the abnormal value. Alternatively, a massage may be displayed upon the imaging to indicate unsuccessful video acquisition, and then the imaging is performed again to acquire appropriate pulse wave information. Alternatively, a pulse wave acquired after a predetermined period of time from the start of the imaging may be used to derive the amplitude. Alternatively, the amplitude may be derived by excluding any abnormal value from a pulse wave acquired during a predetermined period of time. As described above, various methods can be applied to the derivation of the amplitude.

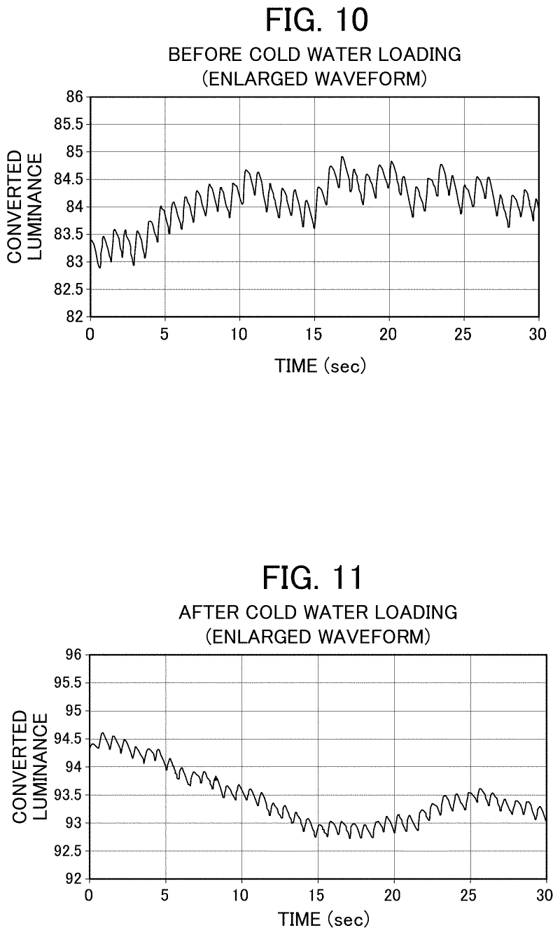

Referring to FIGS. 10 and 11, the following describes specific examples of the pulse wave amplitude. FIG. 10 is a graph showing, in an enlarged manner, the waveform of the temporal change in the converted luminance before the cold water loading as measured using the electronic device 1 according to the one embodiment of the present invention. FIG. 11 is a graph showing, in an enlarged manner, the waveform of the temporal change in the converted luminance after the cold water loading as measured using the electronic device 1 according to the one embodiment of the present invention.

FIG. 10 corresponds to the graph in FIG. 7, and FIG. 11 corresponds to the graph in FIG. 8. The scale for the converted luminance in FIG. 10 is 82 to 86, and the scale for the converted luminance in FIG. 11 is 92 to 96, both of which have a range width of 4. In the graphs shown in FIGS. 10 and 11, each beat in the pulse wave information can be observed. A comparison between FIGS. 10 and 11 indicates that the pulse wave amplitude decreased after the cold water loading.

Cooling a hand is expected to reduce blood flow. In this connection, the cold loading resulted in an increase in the baseline and a decrease in the pulse wave amplitude.

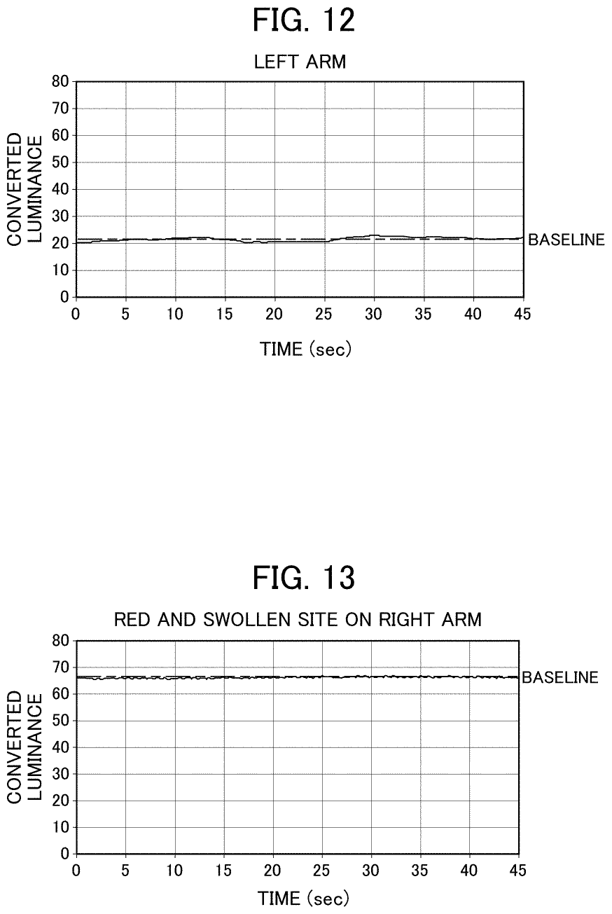

Referring to FIGS. 12 to 15, the following describes another specific exemplary event that is not the cool water loading. With respect to the event described below, target sites on a subject's left and right arms were measured to obtain results. This subject had received vaccination against influenza on the right arm the day before the measurement, and the site given the vaccination was red and swollen.

Trends of the baseline will be described. FIG. 12 is a graph showing temporal change in the converted luminance of the target site on the left arm as measured using the electronic device 1 according to the one embodiment of the present invention. FIG. 13 is a graph showing temporal change in the converted luminance of the target site on the right arm given the vaccination. A comparison between FIGS. 12 and 13 indicates that there was a significant increase in the baseline of the target site on the right arm given the vaccination compared to the baseline of the target site on the left arm.

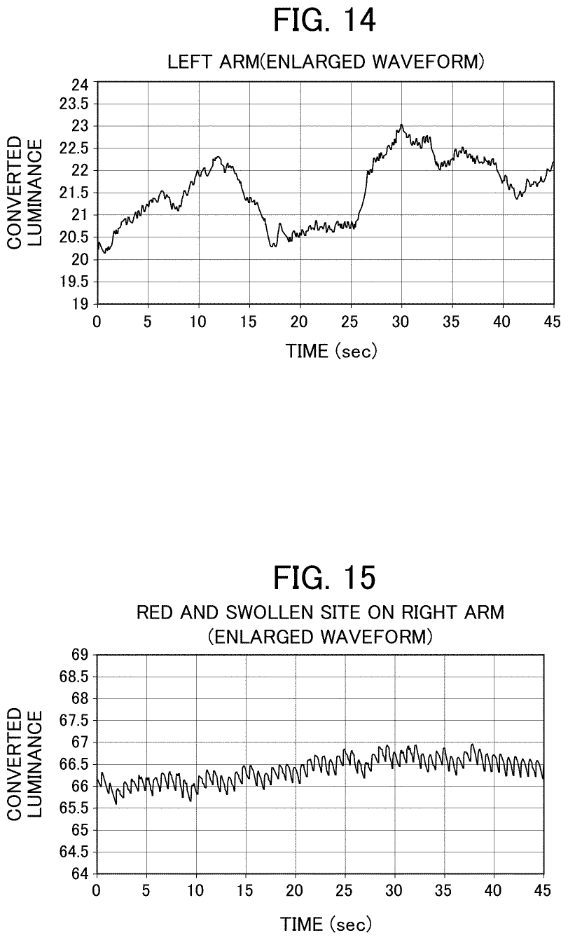

Trends of the pulse wave amplitude will be described. FIG. 14 is a graph showing, in an enlarged manner, the waveform of the temporal change in the converted luminance of the target site on the left arm as measured using the electronic device 1 according to the one embodiment of the present invention. FIG. 15 is a graph showing, in an enlarged manner, the waveform of the temporal change in the converted luminance of the target site on the right arm. A comparison between FIGS. 14 and 15 indicates that there was a significant increase in the pulse wave amplitude of the target site on the right arm given the vaccination compared to the pulse wave amplitude of the target site on the left arm.

These results show that the target site on the right arm, which was red and swollen because of the vaccination, had a significant increase in the baseline and an increase in the pulse wave amplitude compared to the target site on the left arm, which was not red or swollen.

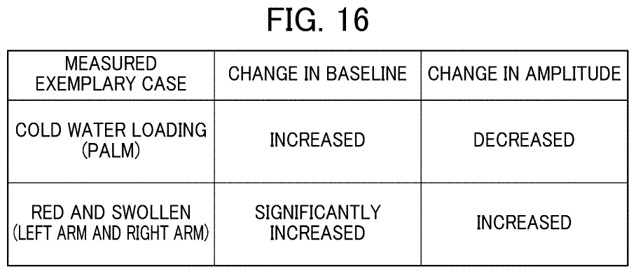

FIG. 16 is a table showing results of the comparisons conducted by the electronic device according to the one embodiment of the present invention between before and after the cold water loading, and between the target site with the vaccination and the target site without the vaccination. As shown in the table in FIG. 16, the cold water loading resulted in an increase in the baseline and a decrease in the pulse wave amplitude, while the red and swollen target site, which is another exemplary case, resulted in a significant increase in the baseline and an increase in the pulse wave amplitude. That is, increasing trends of the baseline and the pulse wave amplitude vary from hemodynamic state to hemodynamic state and are not necessarily consistent with each other.

Conventional technologies such as laser Doppler blood flowmeters and laser speckle blood flowmeters can only derive a single value, i.e., a blood flow rate, of a target measurement site. By contrast, the electronic device 1 according to the present embodiment can derive two different values, i.e., a change in the baseline and a change in the pulse wave amplitude. As such, beyond merely allowing for estimation of an increase or decrease in the blood flow rate, the electronic device 1 allows for estimation of more detailed hemodynamic states.

### [Hemodynamic State Identification]

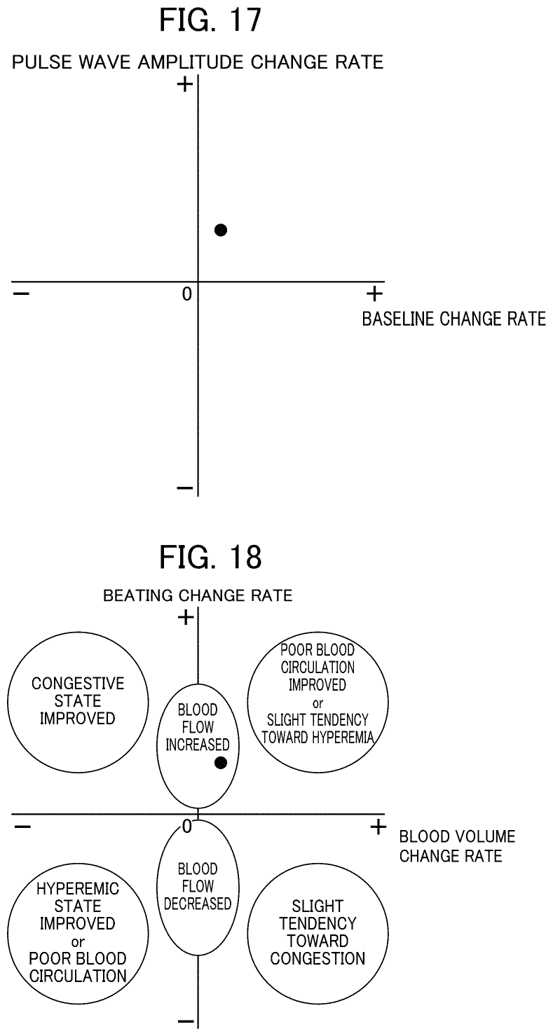

The following describes a method for identifying a hemodynamic state using the baseline and the pulse wave amplitude. FIG. 17 is a graph based on measurement results from the electronic device 1 according to the one embodiment of the present invention. The horizontal axis therein represents baseline change rate (baseline change index), and the vertical axis therein represents pulse wave amplitude change rate (pulse wave amplitude change index). The baseline change rate can be derived in accordance with Equation 1 shown below, and the pulse wave amplitude change rate can be derived in accordance with Equation 2 shown below. It should be noted that a black dot in FIG. 17 is an example of results of Equations 1 and 2 plotted on this map.

Baseline change rate=(BL2/BL1)−1 (Equation 1)
BL1: Baseline in pulse wave information in first measurement
BL2: Baseline in pulse wave information in second measurement

Pulse wave amplitude change rate=(PA2/PA1)−1 (Equation 2)
PA1: Average of pulse wave amplitude values measured for n seconds in first measurement
PA2: Average of pulse wave amplitude values measured for n seconds in second measurement

The following now discusses the meaning of the baseline and the meaning of the pulse wave amplitude. As mentioned above, the principle of extracting a pulse wave from the luminance of video is to capture temporal change in the luminance of green light that is absorbed by hemoglobin. The baseline is therefore considered to be approximately proportional to the average hemoglobin content of the target site during the period of the measurement. That is, a change in the baseline can be interpreted as a change in the average blood volume in the measurement site. By contrast, the pulse wave amplitude itself indicates the beating of the pulse. That is, a change in the pulse wave amplitude can be interpreted as a change in the beating strength.

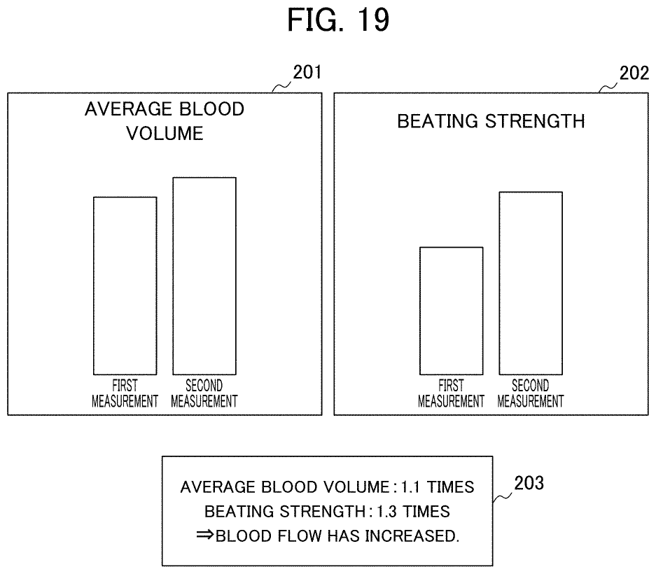

FIG. 18 is a graph showing criteria for identifying a hemodynamic state using the electronic device 1 according to the one embodiment of the present invention. FIG. 18 is a map showing hemodynamic states. FIG. 18 is obtained by changing the horizontal axis in FIG. 17 from baseline change rate to blood volume change rate and changing the vertical axis from pulse wave amplitude change rate to beating change rate. A change in the blood flow can be estimated from a change in the blood volume and a change in the beating.

The following describes hemodynamic states to be identified based on the graph shown in FIG. 18. In this example, after all measurements have been completed, the identification processing unit 115 sets an x-coordinate on the horizontal axis representing level (degree) of the blood volume change rate based on the derived baseline change rate. The data processing unit 114 also sets a y-coordinate on the vertical axis representing level (degree) of the beating change rate. The data processing unit 114 then plots derived results in (x,y) coordinates by locating a derived base change rate on the x-coordinate and a derived pulse wave amplitude change rate on the y-coordinate. The identification processing unit 115 identifies a hemodynamic state based on the plotted position.

For example, if there is almost no change in the blood volume and there is an increase in the beating, the results are plotted in an upper center position shown by a black dot in FIG. 18. In this case, an increased blood flow can be identified as a hemodynamic state. If there is almost no change in the blood volume and there is a decrease in the beating, a decreased blood flow can be identified as a hemodynamic state. For example, the identification processing unit 115 determines that there is almost no change (little change) in the blood volume if the baseline change rate is within a near-1 range of 1 or around 1. In this case, the near-1 range is a numerical range preset based on experience, actual measurement values, or the like.

If there is an increase both in the blood volume and in the beating, the results are plotted in a predetermined range of the upper right first quadrant. In this case, if the first measurement suggests a poor blood circulation state, it is assumed in the second measurement that the poor blood circulation has been improved. If the first measurement suggests a normal state in the above-described case, it is assumed in the second measurement that the measurement site has a slight tendency toward hyperemia. The term “predetermined range” as used in the present description means a range that can be defined by numerical values or a mathematical formula. The identification processing unit 115 may also identify a hemodynamic state based on whether or not the plotted position is within a predetermined range.

It should be noted that a method to be employed for determining whether the measurement site is in a poor blood circulation state or a normal state can be determined as appropriate. For example, the identification processing unit 115 may make such a determination by determining whether or not a measurement value of the pulse wave information, such as the baseline or the pulse wave amplitude, acquired from the first video is greater than a preset threshold value, or by comparing the measurement value against a corresponding past measurement value of the user.

If there is a decrease in the blood volume and there is an increase in the beating, the results are plotted in a predetermined range of the upper left second quadrant. In this case, it is assumed that a congestive state has been improved.

If there is a decrease both in the blood volume and the beating, the results are plotted in a predetermined range of the lower left third quadrant. In this case, if the first measurement suggests a slight tendency toward hyperemia, it is assumed in the second measurement that the hyperemic state has been improved. If the first measurement suggests a normal state in the above-described case, it is assumed in the second measurement that the measurement site has a slight tendency toward poor blood circulation.

It should be noted that a method to be employed for determining whether the measurement site has a slight tendency toward hyperemia or is in a normal state can be determined as appropriate. For example, the identification processing unit 115 may make such a determination by determining whether or not a measurement value of the pulse wave information, such as the baseline or the pulse wave amplitude, acquired from the first video is greater than a preset threshold value, or by comparing the measurement value against a corresponding past measurement value of the user.

If there is an increased in the blood volume and there is a decrease in the beating, the results are plotted in a predetermined range of the lower right fourth quadrant. In this case, it is assumed that the measurement site has a slight tendency toward congestion.

As described above, beyond merely allowing for numerical estimation of blood flow, the electronic device 1 allows for estimation of hemodynamic states. The display processing unit 112 performs a process for displaying a graph (map) such as shown in FIG. 18 on the output unit 18 as a measurement result.

The display processing unit 112 may perform a process for displaying information shown in FIG. 19 along with the information shown in FIG. 18. FIG. 19 is a diagram showing an example of a measurement result (image) displayed on the output unit 18 of the electronic device 1 according to the one embodiment of the present invention. In the image shown in FIG. 19, a frame 201 shows the average blood volume in the first and second measurements in a bar graph, and a frame 202 shows the beating in the first and second measurements in a bar graph. Below the frame 201 and the frame 202 in the image, text 203 is displayed showing messages such as “Average blood volume: 1.1 times”, “Beating strength: 1.3 times”, and “Blood flow has increased”.

The display processing unit 112 generates an image such as shown in FIG. 19 and performs the process for displaying the image on the output unit 18 independently or along with the image of a graph (map) such as shown in FIG. 18.

It should be noted that in a case where the baseline change rate or the pulse wave amplitude change rate is an abnormal value that exceeds a range set for the graph, the identification processing unit 115 may determine that a hemodynamic state cannot be identified properly. For example, the identification processing unit 115 may determine that the measurement site is in an abnormal state if the measurement site is in a normal state in the first measurement, and the baseline change rate or the pulse wave amplitude change rate is greater than or equal to 3. In this case, the display processing unit 112 may be configured to notify the user of the abnormality by performing a process for displaying, on the output unit 18, a message indicating that the electronic device 1 has failed to properly identify a hemodynamic state.

### [Flow of Measurement Processing]

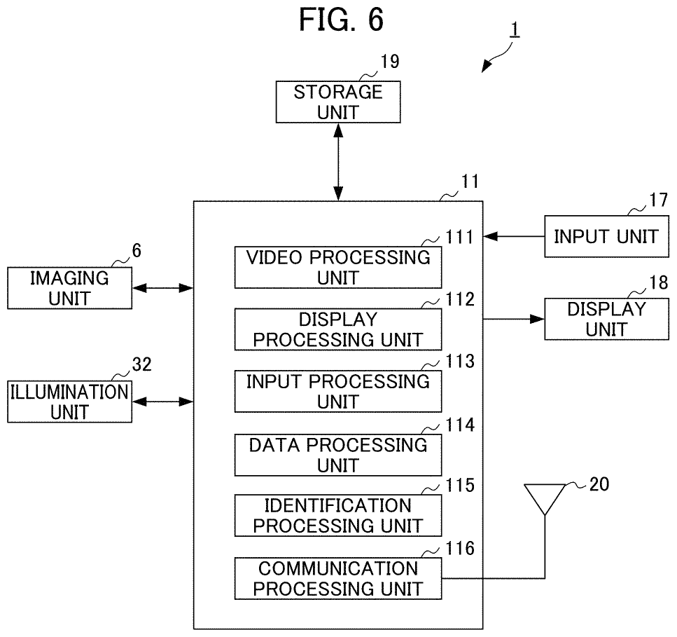

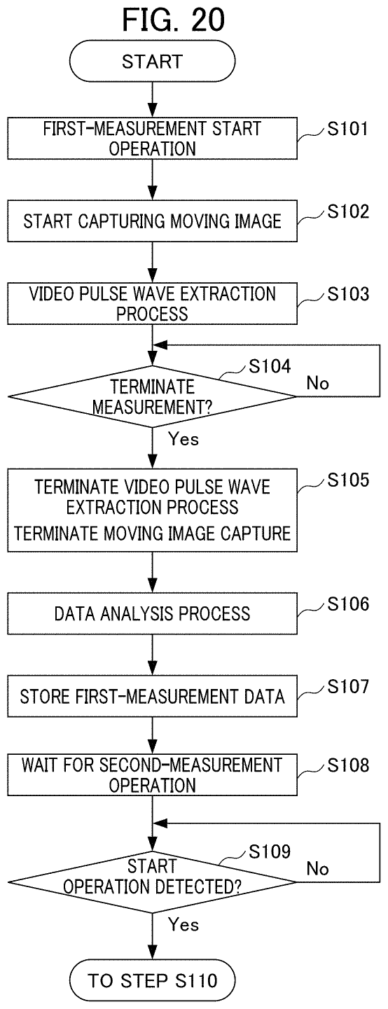

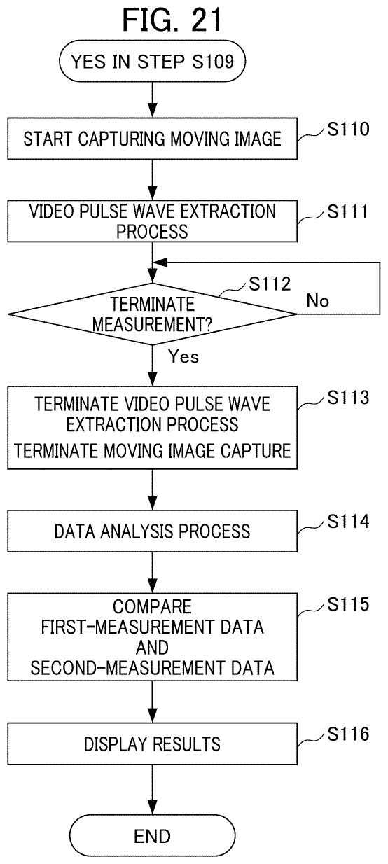

Referring to FIGS. 20 and 21, the following describes a flow of the measurement processing. FIGS. 20 and 21 are each a flowchart for describing the flow of the measurement processing that is performed by the electronic device 1 shown in FIG. 1 having the functional configuration shown in FIG. 6.

As shown in FIG. 20, upon receiving information indicating that the user has performed an operation of starting the first measurement via the input unit 17, the input processing unit 113 transmits, to the video processing unit 111, an instruction to start capturing a moving image (Step S101).

Upon receiving the start instruction from the input processing unit 113, the video processing unit 111 starts capturing a first-measurement moving image including the measurement site using the imaging unit 6 (Step S102). Next, the video processing unit 111 performs a process for extracting first-measurement video pulse wave (pulse wave information) (Step S103).

Next, the video processing unit 111 determines whether or not a condition for terminating the measurement is met (Step S104). The condition for terminating the measurement is, for example, whether or not the image capture has continued for a preset period of time. If the condition for terminating the measurement is not met, the video processing unit 111 continues the image capture until the condition is met (No in Step S104). If the condition for terminating the measurement is met, the video processing unit 111 advances the processing to Step S105 (Yes in Step S104).

In Step S105, the video processing unit 111 terminates the video pulse wave extraction process and the moving image capture using the imaging unit 6 (Step S105). Once the video pulse wave extraction process and the moving image capture have been terminated, the data processing unit 114 performs a process for analyzing first-measurement data acquired by the video processing unit 111 for identification of a hemodynamic state (Step S106). Next, the data processing unit 114 stores, in the storage unit 19, data including results of the first measurement (Step S107).

Upon the data including the results of the first measurement being stored in the storage unit 19, the input processing unit 113 performs a process for waiting for a second-measurement operation (Step S108). As a result of this process, the electronic device 1 becomes ready to receive a second-measurement start operation via the input unit 17. The input processing unit 113 waits for a determination on whether or not the start operation has been detected (Step S109). The input processing unit 113 remains in an operable state until the start operation is detected (No in Step S109). If the start operation has been detected, the input processing unit 113 advances the processing to Step S110 in FIG. 21 (Yes in Step S109).

In Step S110, the video processing unit 111 starts capturing a second-measurement moving image including the measurement site using the imaging unit 6 (Step S110). Next, the video processing unit 111 performs a process for extracting video pulse wave (pulse wave information) from the second-measurement moving image (Step 3113).

Next, the video processing unit 111 determines whether or not a condition for terminating the measurement is met (Step S112). The condition for terminating the measurement is, for example, whether or not the image capture has continued for a preset period of time. If the condition for terminating the measurement is not met, the video processing unit 111 continues the image capture until the condition is met (No in Step S112). If the condition for terminating the measurement is met, the video processing unit 111 advances the processing to Step S113 (Yes in Step S112).

In Step S113, the video processing unit 111 terminates the video pulse wave extraction process and the moving image capture using the imaging unit 6 (Step S113). Once the video pulse wave extraction process and the moving image capture have been terminated, the data processing unit 114 performs a process for analyzing second-measurement data acquired by the video processing unit 111 for identification of a hemodynamic state (Step S114).

The identification processing unit 115 compares the data including the results of the first measurement stored in Step S107 and the data including the results of the second measurement to identify a hemodynamic state (Step S115). According to the present embodiment, the identification processing unit 115 identifies a hemodynamic state by plotting the measurement results on the graph shown in FIG. 18 based on the baseline change rate and the pulse wave amplitude change rate derived by the data processing unit 114.

After completion of the process in Step S115, the display processing unit 112 performs a process for displaying the measurement results including the identified hemodynamic state on the output unit 18 to present the measurement results to the user (Step S116). For example, the information shown in FIGS. 18 and 19 is displayed on the output unit 18. Through the series of processes described above, the user can know his/her own hemodynamic state.

The following describes an experiment aimed to estimate a hemodynamic state and conducted by intentionally setting up a situation that reliably causes a change in hemodynamic state. In this experiment, a measurement using the electronic device 1 according to the present embodiment and a measurement using a two-dimensional laser blood flowmeter according to conventional technology were compared.

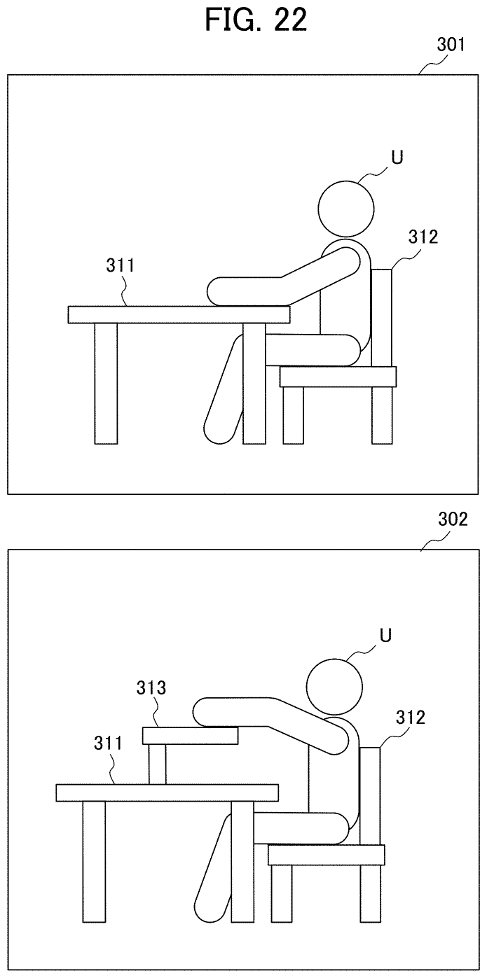

Referring to FIG. 22, the situation set up for this experiment that causes a change in hemodynamic state will be described. In an upper frame 301 in FIG. 22, which shows a desk 311 and a subject U sitting in a chair 312, the desk 311 is positioned so that a hand of the subject U is kept at a lower position than the heart of the subject U. By contrast, in a lower frame 302 in FIG. 22, which shows the desk 311 and the subject U sitting in the chair 312, a stand 313 is disposed on the desk 311 and the hand of the subject U is placed on the stand 313 so that the hand is kept at a higher position than the heart of the subject U. The height difference between the lower hand position (state shown in the frame 301) and the higher hand position (state shown in the frame 302) was 30 cm.

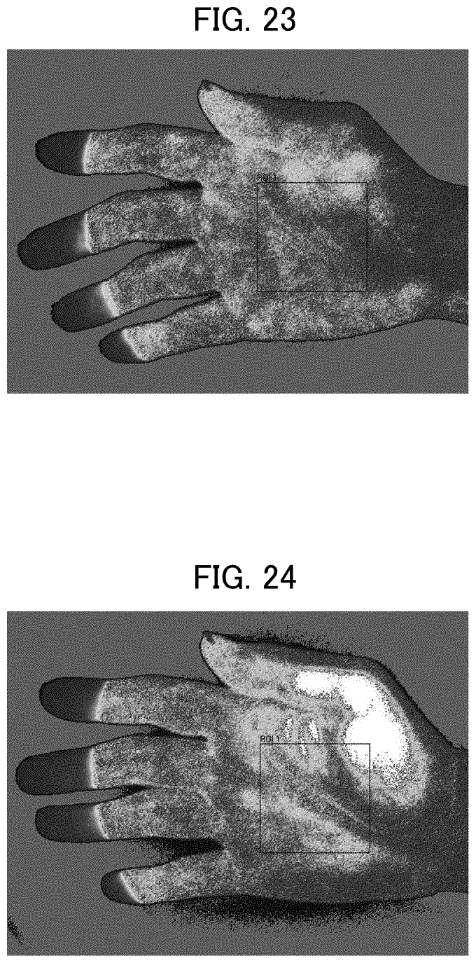

First, a comparative example will be described. FIG. 23 is an image captured when the hand was kept at the lower position and measured using the two-dimensional laser blood flowmeter according to the comparative example. FIG. 24 is an image captured when the hand was kept at the higher position and measured using the two-dimensional laser blood flowmeter according to the comparative example. Rectangular frames in FIGS. 23 and 24 indicate the measurement site (Region of Interest; ROI). Blood flow values measured using the two-dimensional laser blood flowmeter were 18.56 (ml/min/100 g) when the hand was kept at the lower position and 36.67 (ml/min/100 g) when the hand was kept at the higher position. A change rate calculated from these values is 36.67/18.56=1.98.

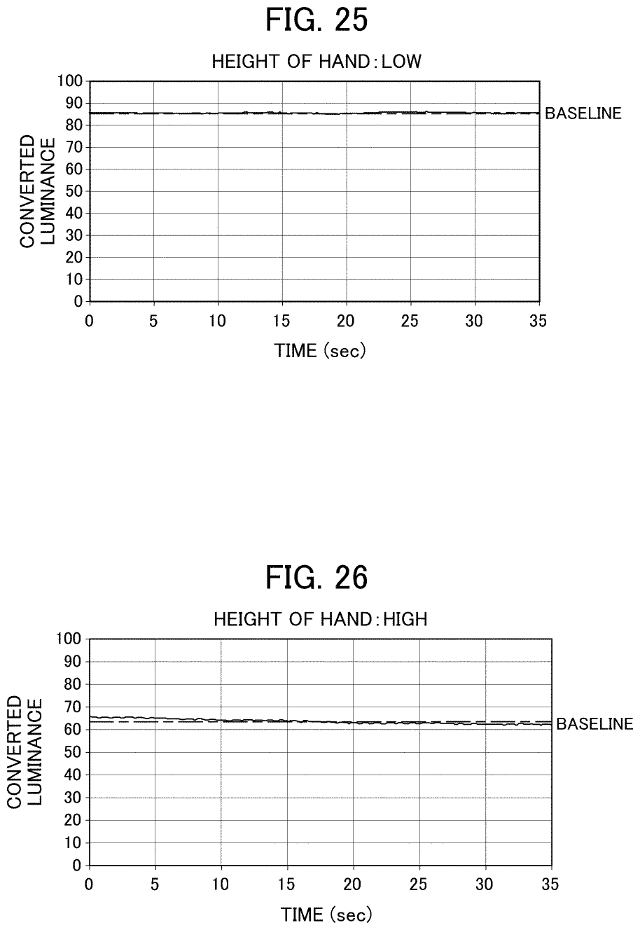

Next, the present embodiment will be described. FIG. 25 is a graph showing temporal change in the converted luminance corresponding to the case where the hand was kept at the lower position and measured using the electronic device 1 according to the one embodiment of the present invention. FIG. 26 is a graph showing temporal change in the converted luminance corresponding to the case where the hand was kept at the higher position and measured using the electronic device 1 according to the one embodiment of the present invention. FIGS. 25 and 26 represent pulse wave information obtained by measuring a measurement site of the palm using the electronic device 1. A comparison between FIGS. 25 and 26 indicates that the baseline is higher when the hand is kept at the lower position.

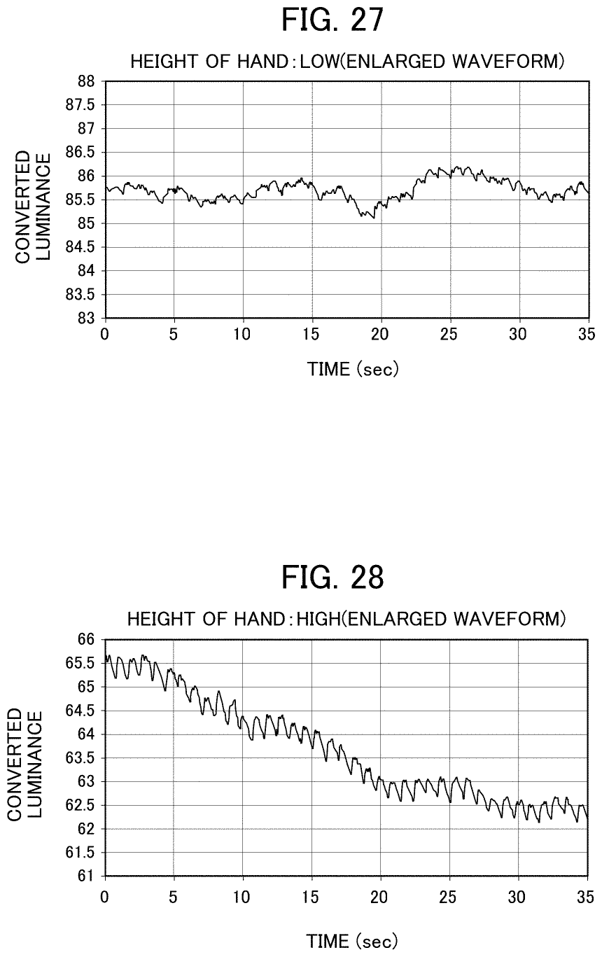

FIG. 27 is a graph showing, in an enlarged manner, the waveform of the temporal change in the converted luminance corresponding to the case where the hand was kept at the lower position and measured using the electronic device according to the one embodiment of the present invention. FIG. 28 is a graph showing, in an enlarged manner, the waveform of the temporal change in the converted luminance corresponding to the case where the hand was kept at the higher position and measured using the electronic device according to the one embodiment of the present invention. The vertical axes of FIGS. 27 and 28 have the same scale range. FIG. 27 is obtained by enlarging FIG. 25 and originated from the same data (converted luminance) as FIG. 25. FIG. 28 is obtained by enlarging FIG. 26 and originated from the same data (converted luminance) as FIG. 26. A comparison between FIGS. 27 and 28 indicates that the pulse wave amplitude is smaller when the hand is kept at the lower position, and the pulse wave amplitude is larger when the hand is kept at the higher position.

The average pulse wave amplitude in FIG. 27 with respect to the hand kept at the lower position was 0.22, and the average pulse wave amplitude in FIG. 28 with respect to the hand kept at the higher position was 0.44. Accordingly, the pulse wave amplitude change rate was 0.44/0.22=2.00, which is significantly close to 1.98 derived as the change rate from the measurement results of the two-dimensional laser blood flowmeter. The results of the experiment have demonstrated that a change in the pulse wave amplitude, which in other words is a change in the beating strength, in the pulse wave information means a change in the blood flow. That is, as shown in FIG. 18, an increase or a decrease in the blood flow can be estimated using an increase or a decrease in the beating change rate.

With the two-dimensional laser blood flowmeter according to the comparative example, it is difficult to measure the average blood volume like the baseline, and therefore it is impossible to capture hemodynamic states such as congestive and hyperemic states. By contrast, with the electronic device 1 according to the present embodiment, a hemodynamic state to be identified when an obviously red and swollen measurement site as in the exemplary case described with reference to FIGS. 12 to 15, which in other words is a measurement site in a hyperemic state, is measured has been verified. It has also been verified that a measurement of a hand in which blood has been intentionally concentrated by varying the height of the hand as in the case shown in FIG. 22, which in other words is a measurement site in a rather congestive state, shows an increase in the baseline and a decrease in the pulse wave amplitude. These verification results support that the electronic device 1 allows for accurate estimation of various blood flow-related states such as shown in FIG. 18.

The following describes effects of the electronic device 1 according to the present embodiment. The electronic device 1 includes the video processing unit 111, the data processing unit 114, and the identification processing unit 115. The video processing unit 111 acquires first pulse wave information (converted luminance) indicating a pulse wave from first video obtained through imaging of at least a specific part of a body, and acquires second pulse wave information (converted luminance) indicating a pulse wave from second video obtained through imaging of the specific part of the body or a part corresponding to the specific part of the body. The data processing unit 114 acquires, from the first pulse wave information and from the second pulse wave information, a baseline, which is an average of pulse wave values acquired during a predetermined period of time, and a pulse wave amplitude, which is an average of pulse wave amplitude values acquired during a predetermined period of time. The data processing unit 114 then derives a baseline change rate (baseline change index), which indicates a change in the baseline between the first pulse wave information and the second pulse wave information, and a pulse wave amplitude change rate (pulse wave amplitude change index), which indicates a change in the pulse wave amplitude between the first pulse wave information and the second pulse wave information. The identification processing unit 115 identifies a hemodynamic state based on a relationship between the baseline change rate and the pulse wave amplitude change rate.

This configuration makes it possible to identify a hemodynamic state based on a change in blood flow that has occurred between the imaging for the first video and the imaging for the second video. Not only an increase or a decrease in blood flow but also hemodynamic states such as congestive and hyperemic states can be identified by deriving the baseline change rate and the pulse wave amplitude change rate with respect to the pulse wave extracted from the video. Furthermore, the above-described configuration, in which the electronic device 1 derives relative changes rather than determining an absolute value of blood flow, makes it possible to identify a hemodynamic state using a general-purpose camera without using specialized equipment such as a laser, allowing for implementation of a system at low cost. Furthermore, the above-described configuration eliminates the need to use a laser, which requires careful handling as in conventional technology, and thus eliminates the need for a specialized operator.

The identification processing unit 115 according to the present embodiment derives the baseline change rate (BL2/BL1) by dividing the baseline (BL2) acquired from the second pulse wave information by the baseline (BL1) acquired from the first pulse wave information, and derives the pulse wave amplitude change rate (PA2/PA1) by dividing the pulse wave amplitude PA2 acquired from the second pulse wave information by the pulse wave amplitude (PA2) acquired from the first pulse wave information.

This configuration makes it possible to identify a hemodynamic state through a simple process of deriving the baseline change rate (BL2/BL1) and the pulse wave amplitude change rate (PA2/PA1), and determining whether or not the baseline change rate (BL2/BL1) and the pulse wave amplitude change rate (PA2/PA1) fall within any of numerical ranges preset as ranges for identifying a hemodynamic state.

Based on the baseline change rate and the pulse wave amplitude change rate, the identification processing unit 115 according to the present embodiment determines that the blood flow has increased if the pulse wave amplitude shows an increasing trend and there is little change in the baseline, and determines that the blood flow has decreased if the pulse wave amplitude shows a decreasing trend and there is little change in the baseline. This configuration makes it possible to accurately determine whether the blood flow is on the increase or on the decrease through a simple process.

Based on the baseline change rate and the pulse wave amplitude change rate, the identification processing unit 115 according to the present embodiment determines that a congestive state has been improved if the pulse wave amplitude shows an increasing trend and there is a decrease in the baseline, and determines that the specific part has a slight tendency toward congestion if the pulse wave amplitude shows a decreasing trend and there is an increase in the baseline. This configuration makes it possible to accurately determine whether or not the specific part is in a congestive state through a simple process.

Based on the baseline change rate and the pulse wave amplitude change rate, the identification processing unit 115 according to the present embodiment determines that poor blood circulation has been improved if there is an increase in the baseline, the pulse wave amplitude shows an increasing trend, and the specific part has been determined to be in a poor blood circulation state based on the first pulse wave information, and determines that the specific part has a slight tendency toward poor blood circulation if there is a decrease in the baseline, the pulse wave amplitude shows a decreasing trend, and the specific part has been determined to be in a normal state based on the first pulse wave information. The identification processing unit 115 determines that a hyperemic state has been improved if there is a decrease in the baseline, the pulse wave amplitude shows a decreasing trend, and the specific part has been determined to have a slight tendency toward hyperemia based on the first pulse wave information, and determines that the specific part has a slight tendency toward hyperemia if there is an increase in the baseline, the pulse wave amplitude shows an increasing trend, and the specific part has been determined to be in a normal state based on the first pulse wave information. This configuration makes it possible to accurately identify a hemodynamic state such as a poor blood circulation state, an improved blood circulation state, or a slight tendency toward hyperemia through a simple process.

The electronic device 1 according to the present embodiment further includes the display processing unit 112 that generates an image showing a measurement result obtained through the identification by the identification processing unit 115. According to this configuration, the image including the measurement result is displayed on the output unit 18, so that the user can easily recognize the measurement result.

The display processing unit 112 generates, as the measurement result, an image in which the baseline change rate and the pulse wave amplitude change rate derived by the data processing unit 114 are plotted on a graph. The graph has vertical and horizontal axes, one of which is set to represent level of the baseline change rate and the other is set to represent level of the pulse wave amplitude change rate, and includes regions respectively showing estimated hemodynamic states. This configuration allows the user to intuitively identify a hemodynamic state. This configuration also allows the user to visually recognize the level of the identified hemodynamic state using the graph.

### Modification Example

The present invention is not limited to the foregoing embodiment, and encompasses changes such as modifications and improvements to the extent that the object of the present invention is achieved. For example, the foregoing embodiment may be modified as described in the following modification example.

The foregoing embodiment is described using the baseline change rate as an example of the baseline change index. However, the operation of subtracting 1 may be omitted. A value obtained by subtracting the baseline in the pulse wave information in the first measurement from the baseline in the pulse wave information in the second measurement may be used as the baseline change index. Likewise, the foregoing embodiment is described using the pulse wave amplitude change rate as an example of the pulse wave amplitude index. However, a value obtained by subtracting the pulse wave amplitude in the pulse wave information in the first measurement from the pulse wave amplitude in the pulse wave information in the second measurement may be used as the pulse wave amplitude change index. As described above, the methods for deriving the baseline change index and the pulse wave amplitude change index may be modified as appropriate.

The foregoing embodiment is described using a configuration in which a comparison process is performed using converted luminance values obtained by converting detected luminance. However, the foregoing embodiment is not limited to this configuration. Such converted luminance values are one form of an indication of the level of luminance, and the conversion process may be omitted from the foregoing embodiment. That is, the comparison process may be performed using the detected luminance values without the conversion process.

For another example, the foregoing embodiment is described on the assumption that the electronic device 1 and each of the servers included in the server group 3 operate in conjunction with each other. However, the functions of the servers may be added to the electronic device 1 so that all processes are performed by the electronic device 1 alone.

The series of processes described above may be executed by hardware or software. The functional configuration described above is merely an example, and no particular limitations are placed thereon. That is, as long as the electronic device 1 has a function of performing the above-described series of processes as a whole, functional blocks to be used for implementing this function is not particularly limited to the examples shown in FIG. 6.

Furthermore, one functional block may be implemented solely by hardware, may be implemented solely by software, or may be implemented by a combination of hardware and software. The functional configuration of the present embodiment is implemented by a processor that executes arithmetic processing. Examples of processors that can be used for the present embodiment include: various independent processing devices such as a single processor, a multi-processor, and a multicore processor; and a combination of any of these various processing devices and a processing circuit such as an application specific integrated circuit (ASIC) or a field programmable gate array (FPGA).

In a case where the series of processes are executed by software, programs forming the software are installed from a network or a recording medium to a computer or the like. The computer may be one incorporated in dedicated hardware. Alternatively, the computer may be one enabled to execute various functions through various programs installed therein, such as a general purpose personal computer.

The recording medium containing the programs includes, for example, the removable medium 100 that is distributed separately from the body of a device in order to provide the programs to each user, or any recording medium that is incorporated in the body of a device and provided to each user along with the device. The removable medium 100 includes, for example, a magnetic disk (including a floppy disk), an optical disk, or a magneto-optical disk. The optical disk includes, for example, compact disk-read only memory (CD-ROM), a DVD, or a Blu-ray (registered trademark) Disc (Blue-ray Disc). The magneto-optical disk includes, for example, a mini-disk (MD). The recording medium that is incorporated in the body of a device and provided to each user along with the device includes, for example, the ROM 12 having programs recorded therein or a hard disk included in the storage unit 19.

It should be noted that writing the programs to be recorded on the recording medium herein includes processes that are not necessarily performed chronologically and that may be performed in parallel or individually as well as processes that are performed chronologically according to the order thereof. The term “system” as used in the present description means an overall apparatus including, for example, a plurality of devices and a plurality of mechanisms.

Although some embodiments of the present invention have been described above, these embodiments are only examples and do not limit the technical scope of the present invention. The present invention can take various other embodiments. Furthermore, various changes such as omissions and substitutions may be made to the present invention to the extent that such changes do not depart from the gist of the present invention. These embodiments and modifications thereof are within the scope and the gist of the invention recited in the present description, and within the scope of the invention recited in the claims and equivalents thereof.

### EXPLANATION OF REFERENCE NUMERALS

1: Electronic device
6: Imaging unit
111: Video processing unit
112: Display processing unit
114: Data processing unit
115: Identification processing unit

1. An electronic device comprising:
at least one processor that executes a program stored in a memory,
wherein the processor is configured to execute processing including
acquiring first pulse wave information indicating a pulse wave from first video obtained through imaging of a specific part of a subject's body during a first period, and acquiring second pulse wave information indicating a pulse wave from second video obtained through imaging of the specific part of the subject's body during a second period later than the first period;
acquiring, from the first pulse wave information and from the second pulse wave information, a baseline of the pulse wave and a pulse wave amplitude, and deriving a baseline change index and a pulse wave amplitude change index, the baseline change index indicating a change in the baseline between the first pulse wave information and the second pulse wave information, and the pulse wave amplitude change index indicating a change in the pulse wave amplitude between the first pulse wave information and the second pulse wave information; and
identifying a hemodynamic state based on a relationship between the baseline change index and the pulse wave amplitude change index.

2. The electronic device according to claim 1, wherein the processor is configured to execute processing comprising:
deriving, as the baseline change index, a baseline change rate by dividing the baseline acquired from the second pulse wave information by the baseline acquired from the first pulse wave information, and
deriving, as the pulse wave amplitude change index, a pulse wave amplitude change rate by dividing the pulse wave amplitude acquired from the second pulse wave information by the pulse wave amplitude acquired from the first pulse wave information.

3. The electronic device according to claim 2, wherein the processor is configured to execute processing comprising:
based on the baseline change rate and the pulse wave amplitude change rate,
determining that blood flow has increased if the pulse wave amplitude shows an increasing trend and there is little change in the baseline, and
determining that the blood flow has decreased if the pulse wave amplitude shows a decreasing trend and there is little change in the baseline.

4. The electronic device according to claim 2, wherein the processor is configured to execute processing comprising:
based on the baseline change rate and the pulse wave amplitude change rate,
determining that a congestive state has been improved if the pulse wave amplitude shows an increasing trend and there is a decrease in the baseline, and
determining that the specific part has a slight tendency toward congestion if the pulse wave amplitude shows a decreasing trend and there is an increase in the baseline.

5. The electronic device according to claim 2, wherein the processor is configured to execute processing comprising:
based on the baseline change rate and the pulse wave amplitude change rate,
determining that poor blood circulation has been improved if there is an increase in the baseline, the pulse wave amplitude shows an increasing trend, and the specific part has been determined to be in a poor blood circulation state based on the first pulse wave information,
determining that the specific part is in a poor blood circulation state if there is a decrease in the baseline, the pulse wave amplitude shows a decreasing trend, and the specific part has been determined to be in a normal state based on the first pulse wave information,
determining that a hyperemic state has been improved if there is a decrease in the baseline, the pulse wave amplitude shows a decreasing trend, and the specific part has been determined to have a slight tendency toward hyperemia based on the first pulse wave information, and
determining that the specific part has a slight tendency toward hyperemia if there is an increase in the baseline, the pulse wave amplitude shows an increasing trend, and the specific part has been determined to be in a normal state based on the first pulse wave information.

6. The electronic device according to claim 1, wherein the processor is configured to execute processing comprising:
generating an image showing a measurement result obtained through the identification by the identification processing unit.

7. The electronic device according to claim 6, wherein the processor is configured to execute processing comprising:
generating, as the measurement result, an image in which the baseline change index and the pulse wave amplitude change index derived by the data processing unit are plotted on a graph, the graph having vertical and horizontal axes, one of which is set to represent level of the baseline change index and the other is set to represent level of the pulse wave amplitude change index, and including regions respectively showing estimated hemodynamic states.

8. A non-transitory computer-readable storage medium for an electronic device for measuring blood flow based on video obtained through imaging of a subject's body, the electronic device including at least one processor, the storage medium storing a program causing the at least one processor to implement functions comprising:
a video processing function of acquiring first pulse wave information indicating a pulse wave from first video obtained through imaging of a specific part of the subject's body during a first period, and acquiring second pulse wave information indicating a pulse wave from second video obtained through imaging of the specific part of the subject's body during a second period later than the first period;
a data processing function of acquiring, from the first pulse wave information and from the second pulse wave information, a baseline of the pulse wave and a pulse wave amplitude, and deriving a baseline change index and a pulse wave amplitude change index, the baseline change index indicating a change in the baseline between the first pulse wave information and the second pulse wave information, the pulse wave amplitude change index indicating a change in the pulse wave amplitude between the first pulse wave information and the second pulse wave information; and
an identification processing function of identifying a hemodynamic state based on a relationship between the baseline change index and the pulse wave amplitude change index.

9. A control method for an electronic device for measuring blood flow based on video obtained through imaging of a subject's body, the control method comprising:
acquiring first pulse wave information indicating a pulse wave from first video obtained through imaging of a specific part of the subject's body during a first period, and acquiring second pulse wave information indicating a pulse wave from second video obtained through imaging of the specific part of the subject's body during a second period later than the first period;
acquiring, from the first pulse wave information and from the second pulse wave information, a baseline of the pulse wave and a pulse wave amplitude, and deriving a baseline change index and a pulse wave amplitude change index, the baseline change index indicating a change in the baseline between the first pulse wave information and the second pulse wave information, the pulse wave amplitude change index indicating a change in the pulse wave amplitude between the first pulse wave information and the second pulse wave information; and
identifying a hemodynamic state based on a relationship between the baseline change index and the pulse wave amplitude change index.
